(19) United States
(12) Patent Application Publication
(10) Pub. No.: US 2020/0046846 A1
(43) Pub. Date: Feb. 13, 2020

(54) MODIFIED OLIGONUCLEOTIDES AND THERAPEUTIC USES THEREOF

(71) Applicants: The Regents of the University of California, Oakland, CA (US); Vybyl Holdings, inc., Woodridge, IL (US)

(72) Inventors: Alexander Roloff, San Diego, CA (US); Nathan C. Gianneschi, San Diego, CA (US); Cassandra E. Callmann, San Diego, CA (US); Matthew P, Thompson, San Diego, CA (US); Paul A. Bertin, Woodridge, IL (US)

(21) Appl. No.: 16/492,642

(22) PCT Filed: Mar. 21, 2018
(86) PCT No.: PCT/US18/23578

§ 371 Date: Sep. 10, 2019

Related U.S. Application Data
(60) Provisional application No. 62/475,185, filed on Mar. 22, 2017.

(51) Int. Cl. A61K 47/68; A61K 47/42; A61K 47/54; A61K 47/64; A61K 9/00; A61P 35/00

(52) U.S. Cl. CPC A61K 47/6807; A61K 47/42; A61P 35/00; A61K 47/643; A61K 9/0019; A61K 47/542

(57) ABSTRACT

The present disclosure generally provides nucleotide-based compounds useful for treating various diseases, including cancer. In some aspects, the disclosure provides oligonucleotides that are chemically modified to include an engineered fatty-acid residue, for example, to assist with improving the half-life of such compounds or assisting with cell penetration (e.g., penetration into tumor cells). In some aspects, the disclosure provides compositions that include such modified nucleotides and a protein, such as albumin or mimetics thereof. The disclosure provides various uses of the compounds and compositions.

### CROSS-REFERENCE TO RELATED APPLICATIONS

The present application claims the benefit of priority to U.S. Provisional Application No. 62/475,185, filed Mar. 22, 2017, which is hereby incorporated by reference as though forth herein in its entirety.

### TECHNICAL FIELD

The present disclosure generally provides nucleotide-based compounds useful for treating various diseases, including cancer. In some aspects, the disclosure provides oligonucleotides that are chemically modified to include an engineered fatty-acid residue, for example, to assist with improving the half-life of such compounds or assisting with cell penetration (e.g., penetration into tumor cells). In some aspects, the disclosure provides compositions that include such modified nucleotides and a protein, such as albumin or mimetics thereof. The disclosure provides various uses of the compounds and compositions.

### DESCRIPTION OF RELATED ART

Oligonucleotides (ONs) represent a class of compounds that offers immense possibilities for treating various diseases. Most ONs operate through some kind of anti-sense mechanism, and are therefore often directed to some kind of RNA species. Examples include, but are not limited to, gapmers, steric block ONs, antagomirs, small interfering RNAs (siRNAs), micro-RNA mimics, and splice switching ONs. In a theoretical sense, such compounds can be used to treat any disorder whose cause is known to be associated with a particular gene. Such diseases include various cancers, diabetes, amyotrophic lateral sclerosis (ALS), Duchenne muscular dystrophy, spinal muscular atrophy, asthma, and arthritis. At present, several ON drugs have received approval from the United States Food and Drug Administration: fomivirsen, for the treatment of cytomegalovirus retinitis; mipomersen, for the treatment of homozygous familial hypercholesterolemia; eteplirsen, for the treatment of Duchenne muscular dystrophy, and nusinersen, for the treatment of spinal muscular atrophy.

Effective delivery and targeting of ON drugs continues to pose a problem. For example, fomivirsen is a synthetic polynucleotide phosphorothioate linkages between the nucleotide units, so as to prevent degradation by nucleases following administration. But such modifications pose their own problems, as such compounds are not readily metabolized into compounds that the body is accustomed to handling. Also, nonspecific binding to various proteins is an issue. Others, such as mipomersen contain black-box warnings because of the risk of off-target side-effects. And many such compounds remain stalled in development because there is no effective means of delivering them to the target tissue. Thus, while ONs offer great promise for treating a myriad of diseases, that hope is yet far from being realized.

Thus, there is a continuing need to develop improved ways of delivering ONs, so as to resist rapid breakdown, reduce off-target side-effects, and/or target particular tissues that are affected by a disease state or condition.

### SUMMARY

The present disclosure provides modified ON compounds and related compositions that can offer one or more of: improved half-life following administration, reduced side-effects from off-target activity, and enhanced targeting of diseased tissue. In some embodiments, the compounds are prodrugs of ONs, such that the prodrug permits improved delivery of the ONs to diseased tissue, such as to a solid cancerous tumor in a mammal. The disclosure also provides methods and uses of those compounds and compositions for the treatment of various diseases, including cancer.

In a first aspect, the disclosure provides compounds of formula (I):

A¹-X¹—X²-A² (I)

wherein: A¹ is an organic group, or is a hydrophilic group, or a hydrogen atom; A² is an oligonucleotide moiety; X¹ is a hydrophobic group; and X² is a direct bond, an organic group, or a heteroatom group selected from the group consisting of —O—, —S—, —S(═O)13 , —S(═O)₂—, —S—S—, —N═, ═N—, —N(H)—, —N═N—N(H)—, —N(H)—N═N—, —N(OH)—, or —N(═O)—. In some embodiments, A¹ is a hydrophilic group, such as a carboxylic acid group (—COOH) or a pharmaceutically acceptable salt thereof. In some embodiments, the hydrophobic group is a C₁₂₋₂₂ hydrocarbylene group, which is optionally substituted. In some embodiments, X² is —O—, —NH—, or an organic group, such as —NH—Z¹—O—C(O)— or —O—Z¹—O—C(O)—, wherein Z¹ is a C₁₋₆ alkylene group that is optionally substituted one or more times by —OH.

In a second aspect, the disclosure provides compositions (e.g., pharmaceutical compositions) that include: a compound of any embodiments of the first aspect; and a protein. In some embodiments, the protein is an albumin or an albumin mimetic.

In a third aspect, the disclosure provides compositions (e.g., pharmaceutical compositions) that include: a compound of any embodiments of the first aspect; a protein, wherein the protein is an albumin or an albumin mimetic; and a carrier, which includes water; wherein the compound and the protein are non-covalently associated with each other; and wherein the compound and the protein are solvated by the carrier.

In a fourth aspect, the disclosure provides methods of treating cancer, which include administering to a subject a compound or composition of any embodiments of any of the foregoing aspects. In some further embodiments thereof, the disclosure provides methods of treating cancer that include administering to a subject one or more immunotherapy agents.

In a fifth aspect, the disclosure provides methods of inducing apoptosis in a cancer cell, which include contacting the cancer cell with a compound or composition of any embodiments of any of the first through the third aspects. In some further embodiments thereof, the disclosure provides methods of inducing apoptosis in a cancer cell that include contacting the cancer cell with one or more immunotherapy agents.

In a sixth aspect, the disclosure provides methods for inhibiting growth of a cancerous tumor, which includes contacting the cancerous tumor with a compound of any embodiments of the first aspect. In some further embodiments thereof, the disclosure provides methods of inhibiting growth of a cancerous tumor that include contacting the cancerous tumor with one or more immunotherapy agents.

In a seventh aspect, the disclosure provides uses of a compound or composition of any embodiments of any of the first through the third aspects as a medicament.

In an eighth aspect, the disclosure provides uses of a compound or composition of any embodiments of any of the first through the third aspects for treating cancer. In some further embodiments thereof, the disclosure provides uses that include use in combination with one or more immunotherapy agents.

In a ninth aspect, the disclosure provides uses of a compound or composition of any embodiments of any of the first through the third aspects in the manufacture of a medicament.

In a tenth aspect, the disclosure provides uses of a compound or composition of any embodiments of any of the first through the third aspects in the manufacture of a medicament for treating cancer.

In an eleventh aspect, the disclosure provides methods of making compounds of the first and second aspects and compositions of the third and fourth aspects.

Further aspects and embodiments are provided in the drawings, the detailed description, the claims, and the abstract.

### BRIEF DESCRIPTION OF DRAWINGS

The following drawings are provided for purposes of illustrating various embodiments of the compounds, compositions, methods, and uses disclosed herein. The drawings are provided for illustrative purposes only, and are not intended to describe any preferred compounds or compositions or any preferred methods or uses, or to serve as a source of any limitations on the scope of the claimed inventions.

FIG. 1 shows a non-limiting example of a compound of formula (I), where the compound includes an oligonucleotide moiety, which is modified to include a long-chain dibasic acid moiety.

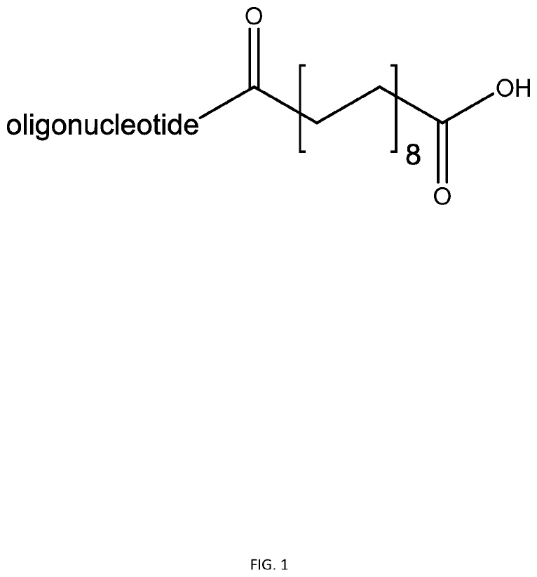

FIG. 2 shows (a) the analytical HPLC trace (top) and (b) MALDI-TOF mass spectrum (bottom) of the purified form of a non-limiting compound of formula (I).

FIG. 3 shows (a) the analytical HPLC trace (top) and (b) MALDI-TOF mass spectrum (bottom) of the purified form of a non-limiting compound of formula (I).

FIG. 4 shows (a) the analytical HPLC trace (left) and (b) MALDI-TOF mass spectrum (right) of the purified form of a non-limiting compound of formula (I).

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “hydrocarbon” refers to an organic group composed of carbon and hydrogen, which can be saturated or unsaturated, and can include aromatic groups. The term “hydrocarbyl” refers to a monovalent or polyvalent (e.g., divalent or higher) hydrocarbon moiety. In some cases, a divalent hydrocarbyl group is referred to as a “hydrocarbylene” group.

As used herein, “alkyl” refers to a straight or branched chain saturated hydrocarbon having 1 to 30 carbon atoms, which may be optionally substituted, as herein further described, with multiple degrees of substitution being allowed. Examples of “alkyl,” as used herein, include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl. In some instances, the “alkyl” group can be divalent, in which case, the group can alternatively be referred to as an “alkylene” group. Also, in some instances, one or more of the carbon atoms in the alkyl or alkylene group can be replaced by a heteroatom (e.g., selected from nitrogen, oxygen, or sulfur, including N-oxides, sulfur oxides, sulfur dioxides, and carbonyl groups, where feasible), and is referred to as a “heteroalkyl” or “heteroalkylene” group, respectively. Non-limiting examples include “oxyalkyl” or “oxyalkylene” groups, which refer to groups where a carbon atom in the alkyl or alkylene group is replaced by oxygen. Non-limiting examples of oxyalkyl or oxyalkylene groups include alkyl or alkylene chains that contain a carbonyl group, and also alkoxylates, polyalkylene oxides, and the like.

The number of carbon atoms in any group or compound can be represented by the terms. Thus, “C_{z}” refers to a group of compound having z carbon atoms, and “C_{x-y}”, refers to a group or compound containing from x to y, inclusive, carbon atoms. For example, “C₁₋₆ alkyl” represents an alkyl group having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. The same logic applies to other types of functional groups, defined below.

As used herein, “alkenyl” refers to a straight or branched chain non-aromatic hydrocarbon having 2 to 30 carbon atoms and having one or more carbon-carbon double bonds, which may be optionally substituted, as herein further described, with multiple degrees of substitution being allowed. Examples of “alkenyl,” as used herein, include, but are not limited to, ethenyl, 2-propenyl, 2-butenyl, and 3-butenyl. In some instances, the “alkenyl” group can be divalent, in which case the group can alternatively be referred to as an “alkenylene” group. Also, in some instances, one or more of the carbon atoms in the alkenyl or alkenylene group can be replaced by a heteroatom (e.g., selected from nitrogen, oxygen, or sulfur, including N-oxides, sulfur oxides, sulfur dioxides, and carbonyl groups, where feasible), and is referred to as a “heteroalkenyl” or “heteroalkenylene” group, respectively.

As used herein, “cycloalkyl” refers to an aliphatic saturated or unsaturated hydrocarbon ring system having 3 to 20 carbon atoms, which may be optionally substituted, as herein further described, with multiple degrees of substitution being allowed. In some embodiments, the term refers only to saturated hydrocarbon ring systems, substituted as herein further described. Examples of “cycloalkyl,” as used herein, include, but are not limited to, cyclopropyl, cyclobutyl, cyclopentyl, cyclohexyl, cyclohexenyl, cycloheptyl, cyclooctyl, adamantyl, and the like. In some instances, the “cycloalkyl” group can be divalent, in which case the group can alternatively be referred to as a “cycloalkylene” group. Cycloalkyl and cycloalkylene groups can also be referred to herein as “carbocyclic rings.” Also, in some instances, one or more of the carbon atoms in the cycloalkyl or cycloalkylene group can be replaced by a heteroatom (e.g., selected independently from nitrogen, oxygen, silicon, or sulfur, including N-oxides, sulfur oxides, and sulfur dioxides, where feasible), and is referred to as a “heterocyclyl” or “heterocyclylene” group, respectively. The term “heterocyclic ring” can also be used interchangeably with either of these terms. In some embodiments, the cycloalkyl and heterocyclyl groups are fully saturated. In some other embodiments, the cycloalkyl and heterocyclyl groups can contain one or more carbon-carbon double bonds.

As used herein, “halogen,” “halogen atom,” or “halo” refer to a fluorine, chlorine, bromine, or iodine atom. In some embodiments, the terms refer to a fluorine or chlorine atom.

As used herein, the terms “organic group,” “organic moiety,” or “organic residue” refer to a monovalent or polyvalent functional group having at least one carbon atom, which optionally contains one or more additional atoms selected from the group consisting of hydrogen atoms, halogen atoms, nitrogen atoms, oxygen atoms, phosphorus atoms, and sulfur atoms, and which does not include covalently bound metal or semi-metal atoms. In some embodiments, these terms can include metal salts of organic groups, such as alkali metal or alkaline earth metal salts of organic anions.

As used herein, the term “pharmacophore” refers to a type of organic functional group. Standard pharmacophores are hydrophobic pharmacophores, hydrogen-bond donating pharmacophores, hydrogen-bond accepting pharmacophores, positive ionizable pharmacophores, and negative ionizable pharmacophores. The classification of organic functional groups within a compound is carried out according to standard classification systems known in the art.

As used herein, the terms “hydrophobic group,” “hydrophobic moiety,” or “hydrophobic residue” refer to an organic group that consists essentially of hydrophobic pharmacophores. In some embodiments, the terms refer to an organic group that consists of hydrophobic pharmacophores.

As used herein, the terms “hydrophilic group,” “hydrophilic moiety,” or “hydrophilic residue” refer to an organic group that comprises one pharmacophores selected from the group consisting of hydrogen bond donors, hydrogen bond acceptors, negative ionizable groups, or positive ionizable groups. In some embodiments, the terms refer to an organic group that consist essentially of pharmacophores selected from the group consisting of hydrogen bond donors, hydrogen bond acceptors, negative ionizable groups, or positive ionizable groups.

As used herein, the term “oligonucleotide moiety” refers to a moiety comprising two or more nucleotide units (generally, from 2 to 200, or from 4 to 100, or from 5 to 50 nucleotide units) linked together. A non-limiting example of such a “oligonucleotide moiety,” is the moiety of the following formula:

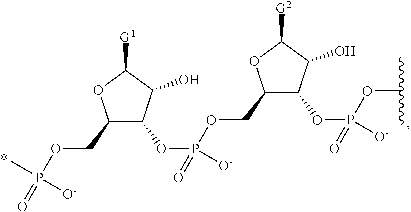

wherein G¹ and G² are base moieties, for example, purine- and pyrimidine-based moieties, including adenine moieties, guanine moieties, cytosine moieties, thymine moieties, and uracil moieties, and wherein the vertical squiggly line indicates that the nucleotide units and phosphodiester linkages continue to the right. Note that the term “oligonucleotide moiety” is not limited to any particular procedure for making such compounds or moieties.

Various methods of drawing chemical structures are used herein. In some instances, the bond line-structure method is used to depict chemical compounds or moieties. In the line-structure method, the lines represent chemical bonds, and the carbon atoms are not explicitly shown (but are implied by the intersection of the lines). The hydrogen atoms are also not explicitly shown, except in instances where they are attached to heteroatoms. Heteroatoms, however, are explicitly shown. Thus, using that methodology, the structures shown below are for 2-methylpropane, 1-methoxypropane, and 1-propanol:

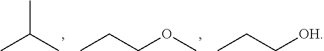

In that methodology, aromatic rings are typically represented merely by one of the contributing resonance structures. Thus, the following structures are for benzene, pyridine, and pyrrole:

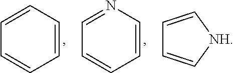

As used herein, a “protein binding moiety” is a moiety that binds non-covalently to one or more sites on a protein with a binding constant (K_{b}) of at least 100 M⁻¹ in water at 25° C.

As used herein, “amino acid” refers to a compound having the structure H₂N—R^{x}—COOH, where Rx is an organic group, and where the NH₂ may optionally combine with Rx (e.g., as in the case of proline). The term includes any known amino acids, including, but not limited to, alpha amino acids, beta amino acids, gamma amino acids, delta amino acids, and the like. In some embodiments, the term can refer to alpha amino acids.

As used herein, “hydroxy acid” refers to a compound having the structure HO—R^{y}—COOH, where R^{y} is an organic group. Non-limiting examples include glycolic acid, lactic acid, and caprolactone.

As used herein, “alkanol amine” refers to a compound having the structure HO—R^{z}—NH₂, where R^{z} is an optionally substituted alkylene group. Non-limiting examples include ethanol amine.

As used herein, “administer” or “administering” means to introduce, such as to introduce to a subject a compound or composition. The term is not limited to any specific mode of delivery, and can include, for example, subcutaneous delivery, intravenous delivery, intramuscular delivery, intracisternal delivery, delivery by infusion techniques, transdermal delivery, oral delivery, nasal delivery, and rectal delivery. Furthermore, depending on the mode of delivery, the administering can be carried out by various individuals, including, for example, a health-care professional (e.g., physician, nurse, etc.), a pharmacist, or the subject (i.e., self-administration).

As used herein, “treat” or “treating” or “treatment” can refer to one or more of: delaying the progress of a disease, disorder, or condition; controlling a disease, disorder, or condition; ameliorating one or more symptoms characteristic of a disease, disorder, or condition; or delaying the recurrence of a disease, disorder, or condition, or characteristic symptoms thereof, depending on the nature of the disease, disorder, or condition and its characteristic symptoms.

As used herein, “subject” refers to any mammal such as, but not limited to, humans, horses, cows, sheep, pigs, mice, rats, dogs, cats, and primates such as chimpanzees, gorillas, and rhesus monkeys. In some embodiments, the “subject” is a human. In some such embodiments, the “subject” is a human who exhibits one or more symptoms characteristic of a disease, disorder, or condition. The term “subject” does not require one to have any particular status with respect to a hospital, clinic, or research facility (e.g., as an admitted patient, a study participant, or the like).

As used herein, the term “compound” includes free acids, free bases, and salts thereof.

As used herein, the term “pharmaceutical composition” is used to denote a composition that may be administered to a mammalian host, e.g., orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques.

Also included within the scope of the disclosure are the individual enantiomers of the compounds represented by Formula (I) or pharmaceutically acceptable salts thereof, as well as any wholly or partially racemic mixtures thereof. The disclosure also covers the individual enantiomers of the compounds represented by Formula (I) or pharmaceutically acceptable salts thereof, as well as mixtures with diastereoisomers thereof in which one or more stereocenters are inverted. Unless otherwise stated, structures depicted herein are also meant to include compounds which differ only in the presence of one or more isotopically enriched atoms. For example, compounds having the present structure, except for the replacement of a hydrogen atom by a deuterium or tritium, or the replacement of a carbon atom by a ¹³C- or ¹⁴C-enriched carbon are within the scope of the disclosure.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, “optionally” means that the subsequently described event(s) may or may not occur. In some embodiments, the optional event does not occur. In some other embodiments, the optional event does occur one or more times.

As used herein, “substituted” refers to substitution of one or more hydrogen atoms of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present. As used herein, the phrases “consist essentially of,” “consists essentially of,” and “consisting essentially of” refer to groups that are open, but which only includes additional unnamed members that would not materially affect the basic characteristics of the claimed subject matter.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, the various functional groups represented will be understood to have a point of attachment at the functional group having the hyphen or dash (-) or a dash used in combination with an asterisk (*). In other words, in the case of —CH₂CH₂CH₃ or *—CH₂CH₂CH₃, it will be understood that the point of attachment is the CH₂ group at the far left. If a group is recited without an asterisk or a dash, then the attachment point is indicated by the plain and ordinary meaning of the recited group.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

Other terms are defined in other portions of this description, even though not included in this subsection.

### Modified Oligonucleotides

In at least one aspect, the disclosure provides compounds of formula (I):

A¹-X¹—X²-A² (I)

wherein: A¹ is a hydrophilic group or a hydrogen atom, or is an organic group; A² is an oligonucleotide moiety; X¹ is a hydrophobic group; and X² is a direct bond, an organic group, or a group selected from the group consisting of —O—, —S—, —S(═O)—, —S(═O)₂—, —S—S—, —N═, ═N—, —N(H)—, —N═N—N(H)—, —N(H)—N═N—, —N(OH)—, or —N(═O)—.

In some embodiments, A¹ is an organic group. A¹ can contain any suitable number of carbon atoms. In some embodiments, for example, A¹ contains from 1 to 100 carbon atoms, or from 1 to 50 carbon atoms, or from 1 to 25 carbon atoms, or from 1 to 10 carbon atoms, or from 1 to 6 carbon atoms. A¹ can also contain one or more heteroatoms, such as nitrogen, oxygen, sulfur, or phosphorus.

In some embodiments according to any of the foregoing embodiments, A¹ is a hydrophilic group or moiety. Non-limiting examples of a hydrophilic group include, but are not limited to, a carboxylic acid moiety, an ester moiety, an amide moiety, a urea moiety, an amine moiety, an ether moiety, an alcohol moiety, a thioether moiety, a thiol moiety, a ketone moiety, an aldehyde moiety, a sulfate moiety, a thiosulfate moiety, a sulfite moiety, a thiosulfite moiety, a phosphate moiety, a phosphonate moiety, a phosphinate moiety, a phosphite moiety, a borate moiety, or a boronate moiety.

In some embodiments of any of the aforementioned embodiments, A¹ is selected from the group consisting of a carboxylic acid group (—COOH), a carboxylate anion (—COO⁻), or a carboxylate ester (—COOR^{a}, where R^{a} is an organic group such as an alkyl or alkoxylate group). In some such embodiments, A¹ is a carboxylic acid group. In some such embodiments, A¹ is a carboxylate ester group.

In some other embodiments of any of the aforementioned embodiments, A¹ is a hydrogen atom. In some other embodiments of any of the aforementioned embodiments, A¹ is a hydroxyl (—OH) group.

In any of the aforementioned embodiments, X¹ can be a hydrophobic group having any suitable number of carbon atoms. In some embodiments, for example, X¹ contains from 1 to 100 carbon atoms, or from 1 to 50 carbon atoms, or from 1 to 25 carbon atoms.

In some embodiments of any of the aforementioned embodiments, X¹ is C₈₋₃₀ hydrocarbylene, which is optionally substituted. In some further embodiments, X¹ is C₁₂₋₂₂ hydrocarbylene, which is optionally substituted. In some further embodiments, X¹ is C₁₂₋₂₂ alkylene. In some further embodiments, X¹ is —(CH₂)₁₂—, —(CH₂)₁₄—, —(CH₂)₁₆—, —(CH₂)₁₈—, —(CH₂)₂₀—, or —(CH₂)₂₂—. In some other embodiments, X¹ is —(CH₂)₁₆—. In some further embodiments, X¹ is C₁₂₋₂₂ alkenylene. In some further such embodiments, X¹ is —(CH₂)₇—CH═CH—(CH₂)₇—.

In some further embodiments of any of the aforementioned embodiments, X¹ is C₁₂₋₂₂ hydrocarbylene, which is optionally substituted. In some such embodiments, X¹ is C₁₂₋₂₂ hydrocarbylene. In some further such embodiments, X¹ is C₁₄₋₂₂ hydrocarbylene. In some further such embodiments, X¹ is C₁₆₋₂₂ hydrocarbylene. In some embodiments of any of the aforementioned embodiments, X¹ is C₁₂₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further such embodiments, X¹ is C₁₄₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further such embodiments, X¹ is C₁₆₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further embodiments of any of the aforementioned embodiments, X¹ is C₁₂₋₂₂ straight-chain alkylene, or C₁₄₋₂₂ straight-chain alkylene, or C₁₆₋₂₂ straight-chain alkylene. In some further embodiments of any of the aforementioned embodiments, X¹ is C₁₂₋₂₂ straight-chain alkenylene, or C₁₄₋₂₂ straight-chain alkenylene, or C₁₆₋₂₂ straight-chain alkenylene.

In some embodiments of any of the aforementioned embodiments, X² is a direct bond. In some other embodiments of any of the aforementioned embodiments, X² is an organic group. In some embodiments, X² is a hydrophilic group. In some embodiments, X² is a heteroalkylene group.

In any of the aforementioned embodiments where X² is an organic group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In any of the aforementioned embodiments where X² is a heteroalkylene group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In some of the aforementioned embodiments, X² can contain certain groups. Some non-limiting examples of such groups that X² can contain are polyalkylene oxide groups, such as polyethylene glycol (PEG) and various polypeptide chains.

In some embodiments, X² is an organic group selected from the group consisting of —C(═O)—, —C(H)═C(H)—, —C(═O)—O—, —O—C(═O)—, —C(═O)—NH—, —NH—C(═O)—, —NH—C(═O)—O—, —O—(C═O)—NH—, —O—C(═O)—O—, —C(═N—NH₂)—, —C(═N—R^{b})— (where R^{b} is a hydrogen atom or an alkyl group), —C(═N—OH)—, —NH—C(═O)—NH—, —NH—C(═S)—NH—, —NH—C(═S)—O—, —O—C(═S)—NH—, —NH—C(═O)—S—, —S—C(═O)—NH—, —NH—C(═S)—S—, —S—C(═S)—NH—, and the cyclic structures shown below:

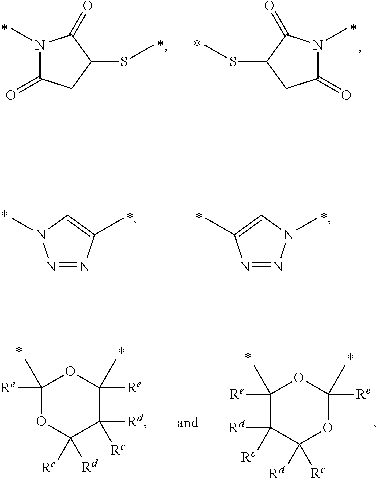

where R^{c}, R^{d}, and R^{e} are, independently at each occurrence, a hydrogen atom or C₁₋₁₀ alkyl. In some further embodiments, X² is —C(═O)—.

In some embodiments, X² is a group selected from the group consisting of —O—, —S—, —S(═O)—, —S(═O)₂—, —S—S—, —N═, ═N—, —N(H)—, —N═N—N(H)—, —N(H)—N═N—, —N(OH)—, and —N(O)—.

In some embodiments, X² comprises one or more moieties selected from the group consisting of: —O—, —NH—, —S—, one or more moieties formed from a alkylene glycols, one or more units formed from alkanol amines, one or more units formed from amino acids, and one or more units formed from hydroxy acids. Thus, in some embodiments, X² comprises one or more moieties formed from alkylene glycols, such as a short poly(ethylene glycol) chain having 1 to 25 ethylene glycol units. In some embodiments, X² comprises one or more moieties formed from amino acids, such as an oligopeptide chain having 1 to 25 amino acid units. In some embodiments, X² comprises one or more moieties formed from hydroxy acids, such as moieties formed from glycolic acid, lactic acid, or caprolactone. In some embodiments, X² comprises a combination of a poly(ethylene glycol) chain having 1 to 25 ethylene glycol units and an oligopeptide having 1 to 25 amino acid units, and optionally one or more units formed from hydroxy acids. In some embodiments, X² is —O—, —S—, —NH—, or an organic group, such as —C(O)—O—Z¹—NH—, —C(O)—O—Z¹—O—, —C(O)—O—Z¹—S—, wherein Z¹ is a C₁₋₆ alkylene group that is optionally substituted one or more times by —OH. In some such embodiments, Z¹ is ethylene. In some such embodiments, Z¹ is —CH₂—CH(OH)—CH₂—.

In any of the above embodiments, the selection of X² will depend on the type of functional group through which it is linked to the oligonucleotide moiety, so as to avoid making compounds that are chemically unstable or impossible. The skilled artisan will be able to select combinations of X² and A² that result in chemically stable compounds, which are compounds in which the chemical structure is not substantially altered when kept at a temperature from about −80° C. to about +40° C., in the absence of moisture or other chemically reactive conditions, for at least a week.

In the above embodiments, A² can be any suitable oligonucleotide moiety, according to the definition set forth above. Such oligonucleotide moieties can contain any suitable number of nucleotide units. In some embodiments, the oligonucleotide moiety comprises from 2 to 200 nucleotide units, or from 3 to 150 nucleotide units, or from 4 to 100 nucleotide units, or from 5 to 50 nucleotide units, or from 6 to 40 nucleotide units.

As used herein, the term “nucleotide unit” refers to a moiety formed from a phosphate-based moiety, a cyclic hydroxy-substituted ether moiety, and a nitrogenous base. In general, the phosphate-based moiety and the nitrogenous base form substituents off of different positions of the cyclic ether group of the cyclic hydroxyl-substituted ether, and in an oligonucleotide moiety, the backbone of the moiety comprises alternating groups formed from phosphate-based moieties and cyclic hydroxyl-substituted ether moieties. Moieties of the formula below represent non-limiting examples of such a nucleotide unit, where G³ is a moiety formed from a nitrogenous base, such as an adenine moiety, a cytosine moiety, a guanine moiety, a thymine moiety, or a uracil moiety:

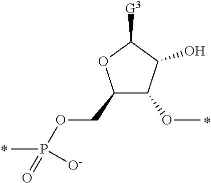

In some embodiments of any of the aforementioned embodiments, the phosphate-based moiety is a phosphate moiety, such as shown above. In some embodiments, one or more of the oxygen atoms can be replaced by sulfur to form phosphorothioate moieties. An example of such a phosphorothioate moiety includes moieties such as —P(═S)(O⁻)—O—. In some other embodiments, the anionic oxygen atom of the phosphate is replaced by an organic group, such as an alkyl or alkyloxy group.

In some embodiments of any of the aforementioned embodiments, the cyclic hydroxy-substituted ether moiety is cyclic ribose moiety (e.g., such as that shown above) or a 2-deoxyribose moiety (where the 2′ position on the ribose is unsubstituted). In both cases, the —OH group at the 1′ position is replaced by the nitrogenous base moiety. In some embodiments, the cyclic hydroxy-substituted ether moiety is a ribose moiety, where the hydroxyl group at the 2′ position is replaced by an organic group, such as a methoxy group, a methoxyethoxy group, or an aminoethoxy group. In some embodiments, the cyclic hydroxy-substituted ether moiety is a ribose moiety, where the hydroxyl group at the 2′ position is replaced by a halogen atom, such as fluorine. In some embodiments, especially where a certain nucleotide unit is the terminal unit in the oligonucleotide chain, the hydroxyl group at the 2′ position is replaced by a nitrogenous base, such as thymine. In some such embodiments, the 3′ position of the ribose or deoxyribose of the terminal nucleotide is a hydroxyl group. In embodiments where the cyclic hydroxy-substituted ether moiety is a ribose moiety, a deoxyribose moiety, or a derivative of either of the foregoing, the oligonucleotide generally forms by linking through the 5′ and 3′ positions, as shown above. In some such embodiments, the —X²—X¹-A¹ moiety conjugates closest to the 5′ position (e.g., via a phosphate-based moiety).

In some embodiments, the nitrogenous base moiety is selected from the group consisting of an adenine moiety, a guanine moiety, a cytosine moiety, a thymine moiety, and a uracil moiety. In some other embodiments, the nitrogenous base can also be selected from certain mimetics of the foregoing, such as dihydrouracil. The adenine and guanine moieties typically connect to the ribose or deoxyribose moiety via the N—H group on the imidazole ring. Examples of nitrogenous base moieties are shown below and on the following page.

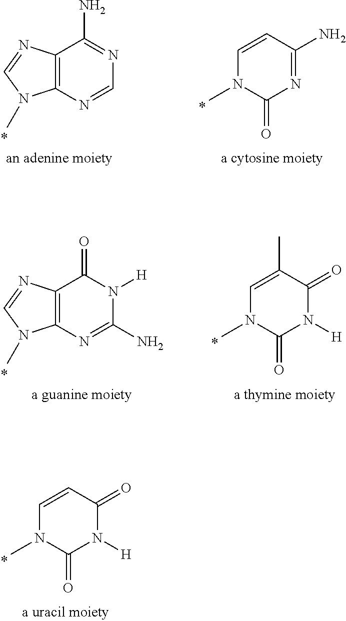

The oligonucleotide moiety according to any of the aforementioned embodiments can be single-stranded or double-stranded. In the double-stranded embodiments, a complementary oligonucleotide is non-covalently bound to the oligonucleotide moiety via hydrogen bonding and/or n-stacking. In such embodiments, the —X²—X¹-A¹ moiety is conjugated to one of the two strands (e.g., the passenger strand), which is non-covalently bound to the other strand (e.g., the guide strand) via hydrogen bonding and/or n-stacking between the base pairs.

The selection of —X²—X¹-A¹ can depend on the nature of the connection to the oligonucleotide moiety.

In embodiments where the —X²—X¹-A¹ connects to a C(═O) group or to a P(═O) group or to a P(═S) group, as is generally the case when linking to oligonucleotide moieties, then —X²—X¹-A¹ is selected from the group consisting of: —O—(CH₂)ₙ₂—C(═O)—OH; —NH—(CH₂)ₙ₂—C(═O)—OH; —NH—(C₁₋₆ alkylene)-O—C(═O)—(CH₂)ₙ₁—C(═O)—OH; —O—(C₁₋₆ alkylene)-O—C(═O)—(CH₂)ₙ₁—C(═O)—OH; —NH—(C₁₋₆ alkylene)-O—C(═O)—(CH₂)ₙ₁—C(═O)—OCH₃; —O—(C₁₋₆ alkylene)-O—C(═O)—(CH₂)ₙ₁—C(═O)—OCH₃; —NH—(C₁₋₆alkylene)-O—C(═O)—(CH₂)ₙ₁—CH₃; —O—(C₁₋₆ alkylene)-O—C(═O)—(CH₂)ₙ₁—CH₃; —NH—(C₁₋₆ alkylene)-C(═O)—O—[(CH₂)₂—O—]ₙ₃(CH₂)ₙ₂—C(═O)—OH; and —O—(C₁₋₆ alkylene)-C(═O)—O—[(CH₂)₂—O—]ₙ₃(CH₂)ₙ₂—C(═O)—OH; wherein n1 is an integer 12 to 24, n2 is an integer from 13 to 25, and n3 is an integer from 1 to 25. In some further such embodiments, —X²—X¹-A¹ is selected from the group consisting of: —O—(CH₂)ₙ₂—C(═O)—OH; —NH—(CH₂)ₙ₂—C(═O)—OH; —NH—(C₁₋₆ alkylene)-O—C(═O)—(CH₂)ₙ₁—C(═O)—OH; —O—(C₁₋₆ alkylene)-O—C(═O)—(CH₂)ₙ₁—C(═O)—OH; —NH—(C₁₋₆ alkylene)-O—C(═O)—(CH₂)ₙ₁—C(═O)—OCH₃; and —O—(C₁₋₆ alkylene)-O—C(═O)—(CH₂)ₙ₁—C(═O)—OCH₃. In some further such embodiments, —X²—X¹-A¹ is selected from the group consisting of: —O—(CH₂)ₙ₂—C(═O)—OH; —NH—(CH₂)ₙ₂—C(═O)—OH; —NH—(C₁₋₆ alkylene)-O—C(═O)—(CH₂)ₙ₁—C(═O)—OH; and —O—(C₁₋₆ alkylene)-O—C(═O)—(CH₂)ₙ₁—C(═O)—OH. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹-A¹ is —O—(CH₂)ₙ₃—OH, where n3 is an integer from 14 to 26, or an integer from 16 to 24, or an integer from 18 to 22.

The compounds described in any of the above embodiments can also exist as pharmaceutically acceptable salts. The term “pharmaceutically acceptable salts” refers to salts of the compounds which are not biologically or otherwise undesirable and are generally prepared by reacting the free base with a suitable organic or inorganic acid or by reacting the acid with a suitable organic or inorganic base. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, there can be formed an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate, and the like.

The compounds above can be made by standard synthetic methods, such as those illustrated in: Sudhir Agrawal, Protocols for Oligonucleotides and Analogs—Synthesis and Properties (Methods in Molecular Biology, Volume 20, 1993, Springer-Verlag New York, LLC); Piet Herdewijn, Oligonucleotide Synthesis: Methods and Applications (Methods in Molecular Biology, Volume 288, 2005, Edition 1, Humana Press); and John Goodchild, Therapeutic Oligonucleotides: Methods and Protocols (Methods in Molecular Biology, Volume 764, 2011, Edition 1, Humana Press, Springer Science+Business Media, LLC). Specific non-limiting examples are shown below in the Examples.

Table 3 (below) shows various examples of compounds that are contemplated by the present disclosure. Table 3 refers to various combinations of an A²-moiety with a —X²—X¹-A¹, which together form compounds of the present disclosure. Table 1 shows illustrative example moieties for the A²-moiety, wherein A² can be the moiety shown or can also be a pharmaceutically acceptable salt thereof. Table 2 shows illustrative example moieties for —X²—X¹-A¹. Table 3 shows non-limiting illustrative combinations of the moieties from Tables 1 and 2, which can come together to form compounds of the present disclosure. The compounds disclosed in Table 3 can be made by methods analogous to those illustrated in the Examples, and by common synthetic methods known to those of ordinary skill in the art. Suitable methods of making such compounds are illustrated in: Sudhir Agrawal, Protocols for Oligonucleotides and Analogs—Synthesis and Properties (Methods in Molecular Biology, Volume 20, 1993, Springer-Verlag New York, LLC); Piet Herdewijn, Oligonucleotide Synthesis: Methods and Applications (Methods in Molecular Biology, Volume 288, 2005, Edition 1, Humana Press); and John Goodchild, Therapeutic Oligonucleotides: Methods and Protocols (Methods in Molecular Biology, Volume 764, 2011, Edition 1, Humana Press, Springer Science+Business Media, LLC).

TABLE 1
A²-Moieties

| | |
|---|---|
| HA1 | anti-survivin siRNA (survivin is an |
| | overexpressed gene in various cancers): |
| | Passenger strand: |
| | 5′-GGACCACCGCAUCUCUACAdTdT-3′ |
| | Guide strand: |
| | 5′-UGUAGAGAUGCGGUGGUCCdTdT-3′ |
| | Or any fluorophore/chromophore-labeled |
| | version thereof, or any fluorophore/ |
| | chromophore-labeled or non-labeled |
| | version thereof containing stabilizing |
| | modifications such as, for example, |
| | phosphorothioates, 2′-fluoro-ribose, |
| | 2′-O-methyl-ribose and others |
| HA2 | microRNA-122 mimic (miR-122 is |
| | suppressed in hepatocellular carcinoma): |
| | 5′-UGGAGUGUGACAAUGGUGUUUG-3′ |
| | Or any fluorophore/chromophore-labeled |
| | version thereof, or any fluorophore/ |
| | chromophore-labeled or non-labeled |
| | version thereof containing stabilizing |
| | modifications such as, for example, |
| | phosphorothioates, 2′-fluoro-ribose, |
| | 2′-O-methyl-ribose and others |
| HA3 | anti-β1 integrin subunit siRNA (integrins |
| | are vital extracellular matrix receptors, |
| | that have been shown to inhibit hepato- |
| | cellular carcinoma growth when knocked-out |
| | (see Bogorad et al., Nat. Commun. |
| | 2014, 5, 3869): |
| | Passenger strand: |
| | 5′-AGAUGAGGUUUAAUUUGAAdTdT-3′ |
| | Guide strand: |
| | 5′-UUCAAAUUGAACCUCAUCUdTdT-3′ |
| | or any fluorophore/chromophore-labeled |
| | version thereof, or any fluorophore/ |
| | chromophore-labeled or non-labeled |
| | version thereof containing stabilizing |
| | modifications such as, for example, |
| | phosphorothioates, 2′-fluoro-ribose, |
| | 2′-O-methyl-ribose and others |
| HA4 | anti-αv integrin subunit siRNA (integrins |
| | are vital extracellular matrix receptors, |
| | that have been shown to inhibit hepato- |
| | cellular carcinoma growth when knocked-out |
| | (see Bogorad et al., Nat. Commun. |
| | 2014, 5, 3869): |
| | Passenger strand: |
| | 5′-GCUUGAAAGAUCAUAAUCAdTdT-3′ |
| | Guide strand: |
| | 5′-UGAUUAUGAUCUUUCAAGCdTdT-3′ |
| | Or any fluorophore/chromophore-labeled |
| | version thereof, or any fluorophore/ |
| | chromophore-labeled or non-labeled |
| | version thereof containing stabilizing |
| | modifications such as, for example, |
| | phosphorothioates, 2′-fluoro-ribose, |
| | 2′-O-methyl-ribose and others |

TABLE 2

| | —X^{2—}X^{1—}A¹ Moieties |
|---|---|
| HB1 | —O—(CH₂)₁₅—C(═O)—OH |
| HB1 | —O—(CH₂)₁₇—C(═O)—OH |
| HB3 | —O—(CH₂)₁₉—C(═O)—OH |
| HB4 | —O—(CH₂)₈—CH═CH—(CH₂)₇—C(═O)—OH |
| HB5 | —NH—(CH₂)₂—O—C(═O)—(CH₂)₁₄—C(═O)—OH |
| HB6 | —NH—(CH₂)₂—O—C(═O)—(CH₂)₁₆—C(═O)—OH |
| HB7 | —NH—(CH₂)₂—O—C(═O)—(CH₂)₁₈—C(═O)—OH |
| HB8 | —NH—(CH₂)₂—O—C(═O)—(CH₂)₇—CH═CH—(CH₂)₇—C(═O)—OH |
| HB9 | —O—(CH₂)₂—O—C(═O)—(CH₂)₁₄—C(═O)—OH |
| HB10 | —O—(CH₂)₂—O—C(═O)—(CH₂)₁₆—C(═O)—OH |
| HB11 | —O—(CH₂)₂—O—C(═O)—(CH₂)₁₈—C(═O)—OH |
| HB12 | —O—(CH₂)₂—O—C(═O)—(CH₂)₇—CH═CH—(CH₂)₇—C(═O)—OH |
| HB13 | —NH—CH₂—C(═O)—O—[(CH₂)₂—O—]₆C(═O)—(CH₂)₁₄—C(═O)—OH |
| HB14 | —NH—CH₂—C(═O)—O—[(CH₂)₂—O—]₆C(═O)—(CH₂)₁₆—C(═O)—OH |
| HB15 | —NH—CH₂—C(═O)—O—[(CH₂)₂—O—]₆C(═O)—(CH₂)₁₈—C(═O)—OH |
| HB16 | —NH—CH₂—C(═O)—O—[(CH₂)₂—O—]₆C(═O)—(CH₂)₇—CH═CH—(CH₂)₇—C(═O)—OH |
| HB17 | —NH—(CH₂)₂—O—C(═O)—(CH₂)₁₄—C(═O)—O—CH₃ |
| HB18 | —NH—(CH₂)₂—O—C(═O)—(CH₂)₁₆—C(═O)—O—CH₃ |
| HB19 | —NH—(CH₂)₂—O—C(═O)—(CH₂)₁₈—C(═O)—O—CH₃ |
| HB20 | —NH—(CH₂)₂—O—C(═O)—(CH₂)₇—CH═CH—(CH₂)₇—C(═O)—O—CH₃ |

TABLE 3

| Com- | | |
|---|---|---|
| pound | A²- | |
| No. | Moiety | —X^{2—}X^{1—}A¹ Moiety |
| 1-20 | HA1 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, respectively |
| 21-40 | HA2 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, respectively |
| 41-60 | HA3 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, respectively |
| 61-80 | HA4 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, respectively |

### Pharmaceutical Compositions

In certain aspects, the compounds of any of the preceding embodiments may be formulated into pharmaceutical compositions in any suitable manner. In general, as compounds for the treatment of cancer, such pharmaceutical formulations are aqueous formulations suitable for parenteral administration, such as intravenous or intra-arterial administration.

In at least one aspect, the disclosure provides pharmaceutical compositions that include one or more compounds of formula (I) (according to any of the foregoing embodiments) and a protein. In some embodiments, the protein is an albumin or an albumin mimetic. In some such embodiments, the protein is human serum albumin (HSA) or a mimetic thereof, i.e., a protein whose sequence is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA. In some embodiments, the protein is human serum albumin.

In certain embodiments of any of the foregoing embodiments, the pharmaceutical composition also includes a carrier, such as a liquid carrier. In some embodiments, the carrier includes water. For example, in some such embodiments, water makes up at least 50% by volume, or at least 60% by volume, or at least 70% by volume, or at least 80% by volume, or at least 90% by volume, based on the total volume of liquid materials in the pharmaceutical composition. The carrier can also include other liquid ingredients, such as liquid ingredients commonly included in aqueous pharmaceutical formulations for parenteral administration.

In certain embodiments having an aqueous carrier, the compounds of formula (I) bind non-covalently to the protein in the pharmaceutical formulation. In some embodiments, the compound of formula (I) and the protein (e.g., human serum albumin) are non-covalently associated with each other with a binding constant (K_{b}) of at least 10² M⁻¹, or at least 10³ M⁻¹, or at least 10⁴ M⁻¹, or at least 10⁵ M⁻¹ at 25° C. in the aqueous composition.

In some embodiments having an aqueous carrier, the compound of formula (I) and the protein are solvated by the carrier. In some such embodiments, at least 90% by weight, or at least 95% by weight, or at least 97% by weight, or at least 98% by weight, or at least 99% by weight of the compounds of formula (I) in the composition are bound non-covalently to the protein with a binding constant (K_{b}) of at least 10² M⁻¹, or at least 10³ M⁻¹, or at least 10⁴ M⁻¹, or at least 10⁵ M⁻¹ at 25° C. in the aqueous composition. In some further such embodiments, the composition is substantially free of agglomerates or nanoparticles. For example, in some embodiments of any of the aforementioned embodiments, no more than 5% by weight, or no more than 4% by weight, or no more than 3% by weight, or no more than 2% by weight, or no more than 1% by weight of the protein-compound (i.e., non-covalently bound conjugates between the protein and one or more compounds of formula (I)) in the aqueous composition have a radius greater than 7 nm, or a radius greater than 5 nm, or a radius greater than 4 nm, as measured by dynamic light scattering.

The compound of formula (I) can have any suitable molar ratio to the protein in the formulation. For example, in some embodiments of any of the foregoing embodiments, the molar ratio of the compound of formula (I) to the protein ranges from 1:10 to 20:1, or from 1:5 to 15:1, or from 1:2 to 10:1. In some embodiments of any of the foregoing embodiments, the molar ratio of the compound of formula (I) to the protein is about 1:1, or is about 2:1, or is about 3:1, or is about 4:1, or is about 5:1, or is about 6:1, or is about 7:1, wherein the term “about,” in this instance means ±0.5:1, such that “about 5:1” refers to a range from 4.5:1 to 5.5:1.

In at least one aspect, the disclosure provides pharmaceutical compositions that include: a compound, which comprises an oligonucleotide moiety and a protein binding moiety; a protein, wherein the protein is an albumin or an albumin mimetic; and a carrier, which comprises water.

In some embodiments, the protein is human serum albumin (HSA) or a mimetic thereof, i.e., a protein whose sequence is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA. In some embodiments, the protein is human serum albumin.

As noted above, in some embodiments, the carrier includes water. For example, in some such embodiments, water makes up at least 50% by volume, or at least 60% by volume, or at least 70% by volume, or at least 80% by volume, or at least 90% by volume, based on the total volume of liquid materials in the pharmaceutical composition. The carrier can also include other liquid ingredients, such as liquid ingredients commonly included in aqueous pharmaceutical formulations for parenteral administration.

In certain embodiments, the compounds bind non-covalently to the protein in the pharmaceutical formulation. In some embodiments, the compound and the protein (e.g., human serum albumin) are non-covalently associated with each other with a binding constant (K_{b}) of at least 10² M⁻¹, or at least 10³ M⁻¹, or at least 10⁴ M⁻¹, or at least 10⁵ M⁻¹ at 25° C. in the aqueous composition.

In some embodiments having an aqueous carrier, the compound and the protein are solvated by the carrier. In some such embodiments, at least 90% by weight, or at least 95% by weight, or at least 97% by weight, or at least 98% by weight, or at least 99% by weight of the compounds of formula (I) in the composition are bound non-covalently to the protein with a binding constant (K_{b}) of at least 10² M⁻¹, or at least 10³ M⁻¹, or at least 10⁴ M⁻¹, or at least 10⁵ M⁻¹ at 25° C. in the aqueous composition. In some further such embodiments, the composition is substantially free of agglomerates or nanoparticles. For example, in some embodiments of any of the aforementioned embodiments, no more than 5% by weight, or no more than 4% by weight, or no more than 3% by weight, or no more than 2% by weight, or no more than 1% by weight of the protein-compound (i.e., non-covalently bound conjugates between the protein and one or more compounds of formula (I)) in the aqueous composition have a radius greater than 7 nm, or a radius greater than 5 nm, or a radius greater than 4 nm, as measured by dynamic light scattering.

The compound of formula (I) can have any suitable molar ratio to the protein in the formulation. For example, in some embodiments of any of the foregoing embodiments, the molar ratio of the compound of formula (I) to the protein ranges from 1:10 to 20:1, or from 1:5 to 15:1, or from 1:2 to 10:1. In some embodiments of any of the foregoing embodiments, the molar ratio of the compound of formula (I) to the protein is about 1:1, or is about 2:1, or is about 3:1, or is about 4:1, or is about 5:1, or is about 6:1, or is about 7:1, wherein the term “about,” in this instance means ±0.5:1, such that “about 5:1” refers to a range from 4.5:1 to 5.5:1.

The pharmaceutical compositions of any of the foregoing aspects and embodiments can also include certain additional ingredients, such as those commonly employed in pharmaceutical compositions for parenteral administration.

### Methods and Uses

The compounds or compositions of any of the foregoing embodiments are useful in the treatment of cancer and related disorders. Therefore, these compounds and compositions can be used for administration to a subject who has or has had a cancerous tumor.

Thus, in certain aspects, the disclosure provides methods of treating cancer, including administering to a subject a compound or composition of any of the foregoing aspects and embodiments. In some embodiments, the subject is a human. In some embodiments, the subject is a subject in need of such treatment, e.g., a human in need of such treatment.

In some aspects, the disclosure provides methods of inducing apoptosis in a cancer cell, including contacting the cancer cell with a compound or composition of any of the foregoing aspects and embodiments.

In some aspects, the disclosure provides methods of inhibiting proliferation of a cancerous tumor, including contacting the cancerous tumor with a compound or composition of any of the foregoing aspects and embodiments.

In some aspects, the disclosure provides uses of a compound or composition of any of the foregoing aspects and embodiments as a medicament.

In some aspects, the disclosure provides uses of a compound or composition of any of the foregoing aspects and embodiments for treating cancer.

In some aspects, the disclosure provides uses of a compound of any of the foregoing aspects and embodiments in the manufacture of a medicament.

In some aspects, the disclosure provides uses of a compound of any of the foregoing aspects and embodiments in the manufacture of a medicament for treating cancer.

### Combination Therapies

The compounds or compositions of any of the foregoing embodiments are useful when used in conjunction with immunotherapy agents, such as checkpoint inhibitors, toll like receptor modulators, and various antibodies, including, but not limited to, alemtuzumab, atezolizumab, ipilimumab, ofatumumab, nivolumab, pembrolizumab, and rituximab.

### EXAMPLES

The following examples show certain illustrative embodiments of the compounds, compositions, and methods disclosed herein. These examples are not to be taken as limiting in any way. Nor should the examples be taken as expressing any preferred embodiments, or as indicating any direction for further research.

The examples may use abbreviations for certain common chemicals. The following abbreviations refer to the compounds indicated.
DMF=Dimethylformamide
DCM=Dichloromethane
NMR=Nuclear magnetic resonance
HPLC=High-performance liquid chromatography
RP-HLPC=Reverse-phase high-performance liquid chromatography
LRMS=Liquid chromatography/low-resolution mass spectrometry
HRMS=Liquid chromatography/high-resolution mass spectrometry
Tips=Triisopropylsilyl
DMAP=4-(Dimethylamino)pyridine
EDC=1-Ethyl-3-(3-dimethylaminopropyl)carbodiimide
THF=Tetrahydrofuran
Dipea=N,N-diisopropylethylamine
HATU=1-[Bis(dimethylamino)methylene]-1H-1,2,3-triazolo-[4,5-b]pyridinium 3-oxide hexafluorophosphate
DCC=N,N′-dicyclohexylcarbodiimide
HSA=Human serum albumin

### Example 1

### Oligonucleotide Example

Solid phase synthesis of oligonucleotides

Oligonucleotides were synthesized on an ABI 394 DNA/RNA synthesizer (Applied Biosystems) in synthesis columns loaded with 1 μmole CPG (1000 Å pore size, Glen Research) bearing the first 5′-Dmt-protected nucleotide. All cyanoethyl phosphoramidites (CEPAs) were purchased from Glen Research: Dmt-dT-CEPA (10-1030) for DNA nucleotides, Dmt-A^{Ac}-TOM-CEPA (10-3004), Dmt-G^{Ac}-TOM-CEPA for RNA nucleotides, and Dmt-2′F-C^{Ac}-CEPA (10-3415) and Dmt-2′F-U-CEPA (10-3430) for 2′F-RNA nucleotides (the presence of a 2′F-pyrimidine is indicated by a superscript ^{F} in the oligonucleotide sequence). 5′-Amino-modifier-5 (10-1905) was used to install a terminal amine on the passenger strand. 3′-Fluorescein-labeled sequences were synthesized on 3′-fluorescein-dT-CPG (20-2056). 4,5-Dicyanoimidazole (DCC) and 5-(benzylthio)-1H-tetrazole (BTT) were used as activators for the synthesis of DNA and RNA/2′F-RNA, respectively. Capping was performed with THF/pyridine/acetic anhydride in acetonitrile (Cap Mix A) and 16% 1-methyl imidazole in THF (Cap Mix B). In each cycle, the phosphorus was oxidized by treatment with 0.02 M iodine in THF/pyridine/water. All 5′-trityl protecting groups were cleaved with 3% trichloroacetic acid in DCM. A coupling cycle consisted of the following steps: detritylation, coupling, capping, and oxidation. Detritylation times were 60 s, coupling times were 30 s for DNA and the 5′-amino-modifier and 180 s for RNA/2′F-RNA. Capping was performed for 5 s, oxidation was carried out for 15 s. All washing and reagent delivery steps were performed as given in the instrument's default synthesis cycle.

Conjugation of Octadecanedioic Acid (ODDA) to the 5′-Terminus of Amine-Modified Nucleic Acids

Before coupling, the terminal Mmt protecting group of the 5′-aminomodifier was cleaved by flushing the support-bound fully protected nucleic acid with 3% trichloroacetic acid in DCM until the yellow color of the Mmt-cation was no longer observable by eye (ca. 3-4.5 min). The support was washed with DCM and acetonitrile and briefly dried under an argon stream. Residual solvent was removed in a desiccator.

Conjugation with small molecules: A solution of 10 equiv. of ODDA-mono-triisopropylsilyl ester (ODDA-TIPS), 9 equiv. HATU and 30 equiv. DIPEA in anhydrous DMF was pre-activated for 5 min and subsequently added to the dried support bearing the 5′-aminomodified nucleic acid sequence. The synthesis column was shaken for 2 h, the support was washed with NMP and DCM and dried in vacuo. The coupling reaction was repeated once with fresh activated ODDA-TIPS (2 h). The support was washed extensively with NMP and DCM to flush away unreacted carboxylic acids and dried in vacuo. The support was stored in a desiccator until the conjugate was cleaved and deprotected.

Release from the Solid Support and Deprotection of Nucleic Acid Conjugates

Release of the CPG-bound oligonucleotides and deprotection of the nucleobases as well as removal of the cyanoethyl protecting groups was carried out by immersing the support in AMA (30% ammonium hydroxide, 40% aqueous methylamine, 1:1, v:v). If a pivaloyl-protected fluorescein dye was present in the sequence, the support was first treated with 30% ammonium hydroxide for 1 h at room temperature to remove the pivaloyl protecting groups before an equal volume of 40% aqueous methylamine was added. AMA solutions containing the solid supports were incubated for 2 h at room temperature to finish deprotection. After centrifugation, the supernatant was removed and the support was washed with 4×200 μL water. The combined solutions were dried down under a stream of dinitrogen (heat should be avoided if 2′F-RNA nucleotides are present in the sequence). To remove 2′TOM protecting groups, the residue was re-dissolved in 115 μL anhydrous DMSO (5 min 65° C.) and 60 μL of anhydrous triethylamine were added. 75 μL triethylamine hydrofluoride complex (NEt₃×3 HF) were added, and the solution was incubated for 2.5 h at 65° C. Afterwards, the solution was briefly cooled in a freezer, and 25 μL of 3 M sodium acetate were added. The oligonucleotide was precipitated with 1 mL of butanol, and incubated for 30 min at −20° C. The suspension was centrifuged for 10 min (12000 rcf) and the supernatant was removed. The precipitate was washed with 2×750 μL ethanol and briefly dried by vacuum centrifugation. The crude oligonucleotide was dissolved in water and analyzed by analytical HPLC and purified by semi-preparative HPLC. The product containing fractions were reduced to ≤10 mL by vacuum centrifugation and the oligonucleotide was desalted using Sep-Pak® C-18 cartridges (Waters). The cartridges were washed with 10 mL acetonitrile and equilibrated with 10 mL water. The oligonucleotides were loaded, washed with 10 mL water, eluted with ca. 6 mL water: acetonitrile (1:1, v:v), dried under reduced pressure and re-dissolved in water. Concentrations were determined via UV-vis spectroscopy and product identity and purity was verified by analytical HPLC and MALDI-TOF-MS. The samples were aliquoted and stored at −20° C. The synthesis scheme is illustrated below.

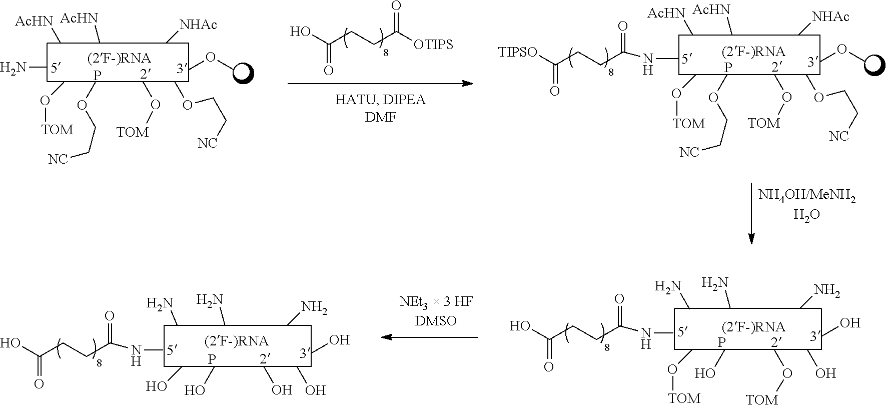

High Performance Liquid Chromatography (HPLC)

HPLC of oligonucleotide samples was performed on a Hitachi Elite LaChrom instrument equipped with phenomenex® clarity 5u Oligo-RP columns (250×10.00 mm for semi-preparative HPLC or 150×4.60 mm for analytical HPLC, 5 micron) at 55° C. Absorbance was measured at 260 nm. Samples were eluted using solvents A (90% 50 mM aqueous triethylammonium acetate (pH 7.0), 10% methanol) and B (methanol) in linear gradients (gradient I: 0% B→80% B in 60 min, flow rate: 4 mL·min⁻¹, gradient II: 0% B→70% B in 60 min, flow rate: 4 mL·min⁻¹, gradient III: 0% B→40% B in 60 min, flow rate: 4 mL·min⁻¹, gradient IV: 0% B→25% B in 60 min, flow rate: 4 mL·min⁻¹, gradient V: 0% B→80% B in 50 min, flow rate: 1 mL·min⁻¹, gradient VI: 0% B→70% B in 50 min, flow rate: 1 mL·min⁻¹, gradient VII: 0% B→40% B in 50 min, flow rate: 1 mL·min⁻¹, gradient VIII: 0% B→30% B in 50 min, flow rate: 1 mL·min⁻¹).

Matrix-Assisted Laser Desorption Ionization Time of Flight Mass Spectrometry (MALDI-TOF-MS)

Mass spectra were recorded at the UCSD Chemistry & Biochemistry Molecular Mass Spectrometry Facility on a Bruker Biflex IV MALDI-TOF instrument in negative mode. A mixture of 2′,4′,6′-trihydroxyacetophenone monohydrate (THAP) and 3-hydroxypicolinic acid (3-HPA) was used as matrices. The 3-HPA matrix was prepared by dissolving 25 mg 3-HPA in 500 μL water/acetonitrile (1:1, v:v) and diluting 454 μL of this solution with 45 μL of 100 mg·ml⁻¹ aqueous diammonium hydrogen citrate. The THAP matrix was prepared by dissolving 15 mg of THAP in 150 μL acetonitrile (saturated solution, solvation was assisted by sonication) and diluting 100 μL of this solution with 100 μL of 23 mg·ml⁻¹ aqueous diammonium hydrogen citrate (obtained by diluting 69 μL 100 mg·ml⁻¹ aqueous diammonium hydrogen citrate with 231 μL water). Oligonucleotide samples were desalted using ZipTip C18 pipette tips (Merck Millipore) prior to MALDI-MS analysis. ZipTips were washed with water/acetonitrile (1:1, v:v, 5×10 μL) and equilibrated with 0.1 M TEAA buffer (5×10 μL), before the oligonucleotide was adsorbed to the ZipTip by aspirating and releasing concentrated stock solutions (10-20×10 μL). The bound oligonucleotide was transferred into the ammonium salt by washing with 0.1 M TEAA buffer (5×10 μL), desalted by washing with water (7×10 μL) and finally released into 2.5 μL THAP matrix. This solution was spotted (1 μL) on top of pre-crystallized 3-HPA matrix (1 μL). The instrument was calibrated with a standard composed of two purchased oligonucleotides spotted on the same target plate. Values are given in mass-to-charge ratios (m/z).

Synthesis and Characterization of Nucleic Acid Conjugates

Survivin siRNA ODDA-Conjugated Passenger Strand with 3′-fluorescein Label:

HOOC—(CH₂)₁₆—CONH—(CH₂)₂—O—(CH₂)₂—OPO₂H-GGAC^{F}C^{F}AC^{F}C^{F}GC^{F}AU^{F}C^{F}U^{F}C^{F}U^{F}AC^{F}AdTdT^{FAM}-3′ (1)

Compound 1 was synthesized on 1 μmol dT^{FAM}-loaded CPG following the general protocol for solid phase synthesis of nucleic acids and purified via preparative HPLC.

Yield: OD_{495 nm}=5.2, 69 nmol, 7%.

ε_{495 nm}=75000 L·mol⁻¹·cm⁻¹, M_{w}=7585.0 g·mol⁻¹.

Analytical HPLC: gradient VI.

MALDI-TOF-MS (m/z): [M-H⁺]⁻: 7581.3 (calculated: 7584.0), [M-2H⁺]²⁻: 3785.9 (calculated: 3791.5).

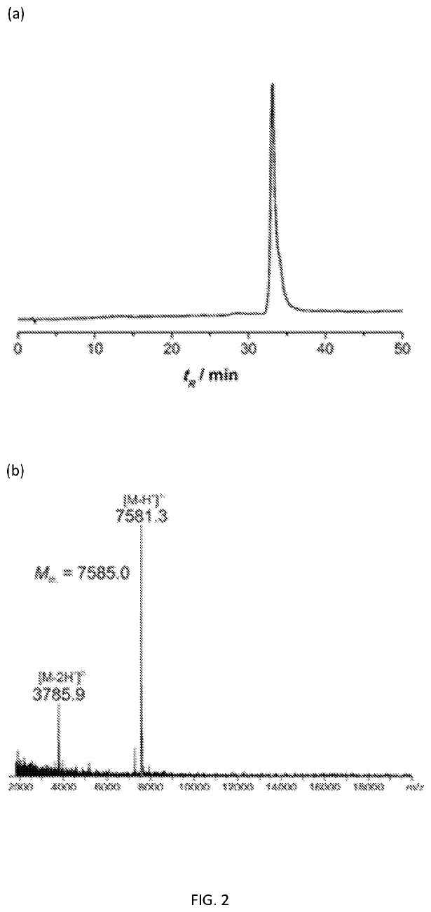

FIG. 2 shows (a) the analytical HPLC trace (top) and (b) MALDI-TOF mass spectrum (bottom) of the purified compound.

Survivin siRNA ODDA-Conjugated Passenger Strand:

HOOC—(CH₂)₁₆—CONH—(CH₂)₂—O—(CH₂)₂—OPO₂H-GGAC^{F}C^{F}AC^{F}C^{F}GC^{F}AU^{F}C^{F}U^{F}C^{F}U^{F}AC^{F}AdTdT-3′ (2)

Compound 2 was synthesized on 1 μmol dT-loaded CPG following the general protocol for solid phase synthesis of nucleic acids. The beads were split in half, and ODDA-TIPS was coupled according to the general protocol. 2 was purified via preparative HPLC.

Yield: OD_{2605 nm}=10.2, 44 nmol, 9%.

ε_{260 nm}=232178 L·mol⁻¹·cm⁻¹, M_{w}=7073.5 g·mol⁻¹.

Analytical HPLC: gradient VI.

MALDI-TOF-MS (m/z): [M-H⁺]⁻: 7070.6 (calculated: 7072.5), [M-2H⁺]²⁻: 3531.4 (calculated: 3535.8).

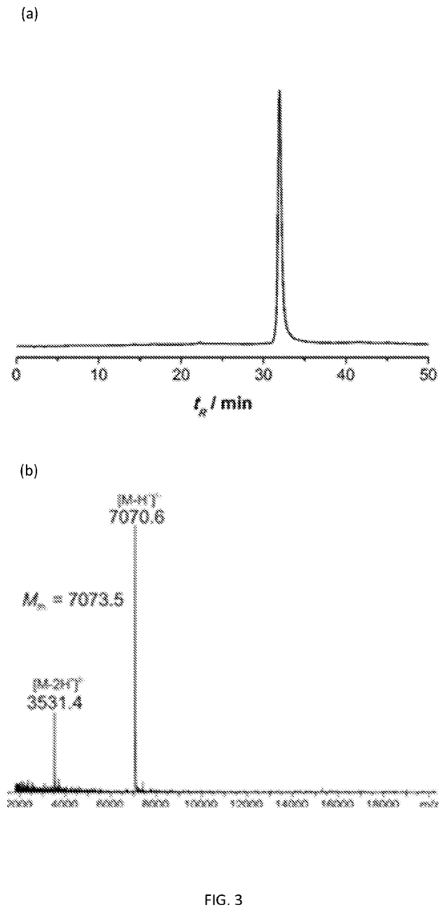

FIG. 3 shows (a) the analytical HPLC trace (top) and (b) MALDI-TOF mass spectrum (bottom) of the purified compound.

Survivin siRNA 5′-Hydroxylated Guide Strand:

5′-U^{F}GU^{F}AGAGAU^{F}GC^{F}GGU^{F}GGU^{F}C^{F}C^{F}dTdT-3′ (3)

3 was synthesized on 1 μmol dT-loaded CPG following the general protocol for solid phase synthesis of nucleic acids. The beads were split in half 3 was purified as 5′-OH RNA via preparative HPLC.

Yield: OD_{260 nm}=16.0, 64 nmol, 6%.

ε_{260 nm}=249575 L·mol⁻¹·cm⁻¹, M_{w}=6758.0 g·mol⁻¹.

Analytical HPLC: gradient VII.

MALDI-TOF-MS (m/z): [M-H⁺]⁻: 6755.8 (calculated: 6757.0), [M-2H⁺]²⁻: 3374.9 (calculated: 3378.0).

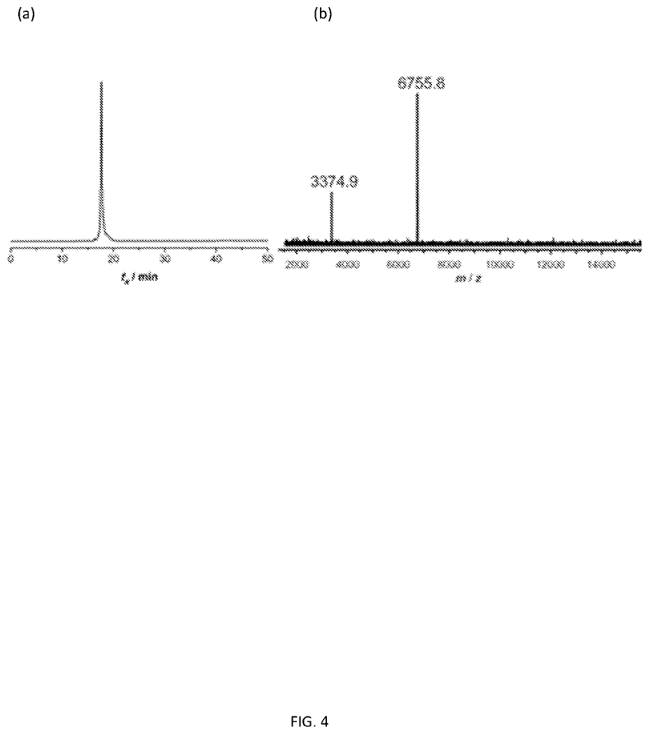

FIG. 4 shows (a) the analytical HPLC trace (left) and (b) MALDI-TOF mass spectrum (right) of the purified compound.

Formulation of ODDA-RNA-HSA Complexes

Single or double-stranded RNAs were dissolved to the desired concentration (for gel shift assays: 10 μM, for circulation time studies: 75 μM) in PBS buffer (1× final concentration) containing freshly constituted HSA (600 μM final concentration). The solutions were incubated overnight at room temperature to ensure equilibration.

1. A compound of formula (I)
A¹-X¹—X²-A² (I)
wherein:
A¹ is an organic group; or A¹ is a hydrophilic group or a hydrogen atom;
A² is an oligonucleotide moiety;
X¹ is a hydrophobic group; and
X² is a direct bond, an organic group, —O—, —S—, —S(═O)—, —S(═O)₂—, —S—S—, —N═, ═N—, —N(H)—, —N═N—N(H)—, —N(H)—N═N—, —N(OH)—, or —N(═O)—.

2. The compound of claim 1, wherein A¹ is a carboxylic acid group, a carboxylate anion, or a carboxylate ester.

3. The compound of claim 2, wherein A¹ is a carboxylic acid group.

4. The compound of any one of claims 1 to 3, wherein the oligonucleotide moiety comprises from 2 to 200 nucleotide units, or from 3 to 150 nucleotide units, or from 4 to 100 nucleotide units, or from 5 to 50 nucleotide units, or from 6 to 40 nucleotide units.

5. The compound of any one of claims 1 to 4, wherein the oligonucleotide moiety conjugates to X² via a phosphate moiety or a thiophosphate moiety.

6. The compound of claim 4 or 5, wherein the nucleotide units comprise ribose moieties or deoxyribose moieties.

7. The compound of any one of claims 4 to 6, wherein the nucleotide units each comprise a nitrogenous base moiety selected from the group consisting of an adenine moiety, a cytosine moiety, a guanine moiety, a thymine moiety, and a uracil moiety.

8. The compound of any one of claims 1 to 7, wherein the oligonucleotide moiety is HA1, HA2, HA3, HA4, or related or nonrelated siRNA, microRNA mimic, or antisense sequences, and pharmaceutically acceptable salts of any of the foregoing.

9. The compound of any one of claims 1 to 8, wherein X¹ is C₁₂₋₂₂ hydrocarbylene, which is optionally substituted.

10. The compound of claim 9, wherein X¹ is C₁₂₋₂₂ alkylene group.

11. The compound of claim 10, wherein X¹ is —(CH₂)₁₂—, —(CH₂)₁₄—, —(CH₂)₁₆—, —(CH₂)₁₈—, —(CH₂)₂₀—, or —(CH₂)₂₂—.

12. The compound of claim 11, wherein X¹ is —(CH₂)₁₆—.

13. The compound of claim 12, wherein X² is —C(O)—O—Z¹—NH—, —C(O)—O—Z¹—O—, or —C(O)—O—Z¹—S—, wherein Z¹ is a C₁₋₆ alkylene group that is optionally substituted one or more times by —OH.

14. The compound of claim 13, wherein Z¹ is ethylene or —CH₂—CH(OH)—CH₂—.

15. A pharmaceutical composition comprising:
a compound of any one of claims 1 to 14; and
a protein, wherein the protein is human serum albumin or a protein whose sequence is at least 50% equivalent to that of human serum albumin.

16. The pharmaceutical composition of claim 15, wherein the protein is human serum albumin.

17. The pharmaceutical composition of claim 15 or 16, further comprising a carrier.

18. The pharmaceutical composition of claim 17, wherein the carrier comprises water.

19. The pharmaceutical composition of claim 18, wherein the compound and the protein are non-covalently associated with each other with a binding constant (K_{b}) of at least 10² M⁻¹, or at least 10³ M⁻¹, or at least 10⁴ M⁻¹, or at least 10⁵ M⁻¹.

20. The pharmaceutical composition of any one of claims 17 to 19, wherein the compound and the protein are solvated by the carrier.

21. The pharmaceutical composition of any one of claims 17 to 20, which contains one or more compounds of any one of claims 1 to 16 and one or more proteins, wherein at least 90% by weight, or at least 95% by weight, or at least 97% by weight, or at least 99% by weight, of the compounds in the composition are bound to proteins with a binding constant (K_{b}) of at least 10² M⁻¹, or at least 10³ M⁻¹, or at least 10⁴ M⁻¹, or at least 10⁵ M⁻¹.

22. The pharmaceutical composition of claim 21, wherein at least at least 90% by weight, or at least 95% by weight, or at least 97% by weight, or at least 99% by weight, of the protein-bound particles in the composition have a radius no greater than 5 nm, or no greater than 4 nm, as measured by dynamic light scattering.

23. The pharmaceutical composition of any one of claims 17 to 22, wherein the pharmaceutical composition is suitable for parenteral administration to a mammal, e.g., a human.

24. The pharmaceutical composition of any one of claims 17 to 22, wherein the pharmaceutical composition is suitable for intravenous administration to a mammal, e.g., a human.

25. A pharmaceutical composition comprising:
a compound, which comprises an oligonucleotide moiety and a protein binding moiety;
a protein, wherein the protein is human serum albumin or a protein whose sequence is at least 50% equivalent to that of human serum albumin; and
a carrier, which comprises water;
wherein the compound and the protein are non-covalently associated with each other with a binding constant (K_{b}) of at least 10² M⁻¹, or at least 10³ M⁻¹, or at least 10⁴ M⁻¹, or at least 10⁵ M⁻¹; and
wherein the compound and the protein are solvated by the carrier.

26. The pharmaceutical composition of claim 25, wherein the compound is a compound of any one of claims 1 to 16.

27. The pharmaceutical composition of claim 25 or 26, wherein the protein is human serum albumin.

28. The pharmaceutical composition of any one of claims 25 to 27, which contains one or more compounds of any one of claims 1 to 16 and one or more proteins, wherein at least 90% by weight, or at least 95% by weight, or at least 97% by weight, or at least 99% by weight, of the compounds in the composition are bound to proteins with a binding constant (K_{b}) of at least 10² M⁻¹, or at least 10³ M⁻¹, or at least 10⁴ M⁻¹, or at least 10⁵ M⁻¹.

29. The pharmaceutical composition of claim 28, wherein at least at least 90% by weight, or at least 95% by weight, or at least 97% by weight, or at least 99% by weight, of the protein-bound particles in the composition have a radius of no greater than 5 nm, or no greater than 4 nm, as measured by dynamic light scattering.

30. The pharmaceutical composition of any one of claims 25 to 29, wherein the pharmaceutical composition is suitable for parenteral administration to a mammal, e.g., a human.

31. The pharmaceutical composition of any one of claims 25 to 29, wherein the pharmaceutical composition is suitable for intravenous administration to a mammal, e.g., a human.

32. A method of treating cancer, comprising:
administering to a subject a compound of any one of claims 1 to 14 or a composition of any one of claims 15 to 31.

33. The method of claim 32, further comprising administering to a subject an immunotherapy agent.

34. The method of claim 33, wherein administering the immunotherapeutic agent to the subject is carried out concurrently with, or within no more than three days before or after, administering to the subject the compound of any one of claims 1 to 14 or the composition of any one of claims 15 to 31.

35. A method of inducing apoptosis in a cancer cell, comprising:
contacting the cancer cell with a compound of any one of claims 1 to 14 or a composition of any one of claims 15 to 31.

36. A method of inhibiting proliferation of a cancerous tumor, comprising:
contacting the cancerous tumor with a compound of any one of claims 1 to 14 or a composition of any one of claims 15 to 31.

37. Use of a compound of any one of claims 1 to 14 or a composition of any one of claims 15 to 31 as a medicament.

38. Use of a compound of any one of claims 1 to 14 or a composition of any one of claims 15 to 31 for treating cancer.

39. Use of a compound of any one of claims 1 to 14 in the manufacture of a medicament.

40. Use of a compound of any one of claims 1 to 14 in the manufacture of a medicament for treating cancer.
